(19) United States
(12) Patent Application Publication
(10) Pub. No.: US 2012/0089024 A1
(43) Pub. Date: Apr. 12, 2012

(54) METHOD OF PROVIDING THREE DIMENSIONAL COLOR DOPPLER IMAGE AND ULTRASOUND SYSTEM FOR IMPLEMENTING THE SAME

(75) Inventor: Sung Hoo HONG, Seoul (KR)

(73) Assignee: SAMSUNG MEDISON CO., LTD.

(21) Appl. No.: 13/252,591

(22) Filed: Oct. 4, 2011

(30) Foreign Application Priority Data
Oct. 8, 2010 (KR) 10-2010-0098137

(51) Int. Cl. A61B 8/08

(52) U.S. Cl. 600/441; 600/453

(57) ABSTRACT

There is disclosed an embodiment for providing a three dimensional color Doppler image. An ultrasound data acquisition unit may transmit and receive ultrasound signals to and from a target object to output ultrasound data. A processor may form Doppler signals including power information of the target object and at least one of direction information and velocity information of the target object by using the ultrasound data. The processor may further form a three dimensional Doppler mode image representing the power information and at least one of the direction information and the velocity information by using the Doppler signals.

### CROSS-REFERENCE TO RELATED APPLICATION

The present application claims priority from Korean Patent Application No. 10-2010-0098137 filed on Oct. 8, 2010, the entire subject matter of which is incorporated herein by reference.

### TECHNICAL FIELD

The present invention generally relates to ultrasound systems, and more particularly to an ultrasound system and method for providing a three dimensional color Doppler image.

### BACKGROUND

An ultrasound system has become an important and popular diagnostic tool due to its non-invasive and non-destructive nature. The ultrasound system can provide high dimensional real-time ultrasound images of inner parts of target objects without any surgical operation.

Generally, the ultrasound system may operate in various image modes such as a brightness mode (B mode), a Doppler mode (D mode), a color Doppler mode (C mode), an elastic mode and the like to acquire the ultrasound images for diagnosis. In the B mode, the ultrasound system can provide a B mode image that visualizes reflection coefficients of ultrasound signals reflected from the target objects as a two dimensional image. In the D mode, the ultrasound system can provide a D mode image that visualizes velocities of moving objects (e.g., blood flow) as Doppler spectra using the Doppler Effect. In the C mode, the ultrasound system can provide a color Doppler mode image that visualizes velocities of the moving objects as colors using the Doppler Effect. In the elastic mode, the ultrasound system can provide an elastic mode image that visualizes response differences between the target objects with and without compression.

In the C mode, the ultrasound system may utilize frequency differences (Doppler frequency) between a frequency (transmit frequency) of the ultrasound signals transmitted from an ultrasound probe as pulse repetition frequency (PRF) and a frequency (receive frequency) of Doppler signals reflected from the moving objects and received through the ultrasound probe. For example, the receive frequency of the moving objects toward the ultrasound probe is higher than the transmit frequency, and the receive frequency of the moving objects away from the ultrasound probe is lower than the transmit frequency.

Conventionally, the C mode image includes a color flow image providing image information such as an existence, a mean velocity and a direction of a blood flow in the region of interest of a two dimensional ultrasound image and a power Doppler image providing information on the existence and a volume of the blood flow by representing a power of the blood flow only instead of the velocity and direction of the blood flow. Thus, it was inconvenient that both the color flow image and the power Doppler image need to be respectively displayed to synthetically observe the direction, velocity and power of the blood flow.

### SUMMARY

An embodiment for providing three dimensional color Doppler images is disclosed herein. In one embodiment, by way of non-limiting example, an ultrasound system may include: an ultrasound data acquisition unit configured to transmit and receive ultrasound signals to and from a target object to output ultrasound data; and a processor coupled to the ultrasound data acquisition unit and configured to form Doppler signals including power information of the target object and at least one of direction information and velocity information of the target object by using the ultrasound data and form a three dimensional Doppler mode image representing the power information and at least one of the direction information and the velocity information by using the Doppler signals.

In another embodiment, a method of providing a three dimensional color Doppler image may comprise: a) transmitting and receiving ultrasound signals to and from a target object to output ultrasound data; b) forming Doppler signals including power information of the target object and at least one of direction information and velocity information of the target object by using the ultrasound data; and c) forming a three dimensional Doppler mode image representing the power information and at least one of the direction information and the velocity information by using the Doppler signals.

In yet another embodiment, there is provided a computer readable medium comprising computer executable instructions configured to perform the following acts: a) transmitting and receiving ultrasound signals to and from a target object to output ultrasound data; b) forming Doppler signals including power information of the target object and at least one of direction information and velocity information of the target object by using the ultrasound data; and c) forming a three dimensional Doppler mode image representing the power information and at least one of the direction information and the velocity information by using the Doppler signals.

This Summary is provided to introduce a selection of concepts in a simplified form that are further described below in the Detailed Description. This Summary is not intended to identify key or essential features of the claimed subject matter, nor is it intended to be used in determining the scope of the claimed subject matter.

### BRIEF DESCRIPTION OF THE DRAWINGS

FIG. 1 is a block diagram showing an illustrative embodiment of an ultrasound system.

FIG. 2 is a schematic diagram showing an example of a brightness mode (B mode) image.

FIG. 3 is a block diagram showing an illustrative embodiment of an ultrasound data acquisition unit.

FIG. 4 is a block diagram showing an illustrative embodiment of a processor.

FIG. 5 is a schematic diagram showing an example of a three dimensional Doppler mode image.

### DETAILED DESCRIPTION

This detailed description is provided with reference to the accompanying drawings. One of ordinary skill in the art may realize that the following description is illustrative only and is not in any way limiting. Other embodiments of the present invention may readily suggest themselves to such skilled persons having the benefit of this disclosure.

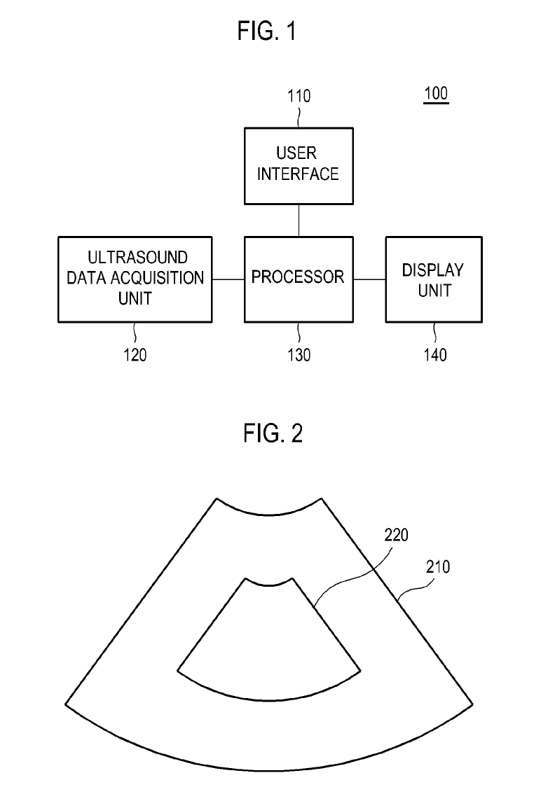

FIG. 1 is a block diagram showing an illustrative embodiment of an ultrasound system. As depicted therein, the ultrasound system 100 may include a user interface 110, an ultrasound data acquisition unit 120, a processor 130 and a display unit 140.

The user interface 110 may receive input information from a user. FIG. 2 is a schematic diagram showing an example of a brightness mode (B mode) ultrasound image. In one embodiment, the input information may include information for setting a region of interest (ROI) 220 on the B mode ultrasound image 210. The ROI 220 may include a color box. The user interface 110 may include a control panel, a mouse, a keyboard and the like.

The ultrasound data acquisition unit 120 may be configured to transmit and receive ultrasound signals to and from a target object to thereby form ultrasound data to be used in forming frames. The frames may include color Doppler mode (C mode) image frames.

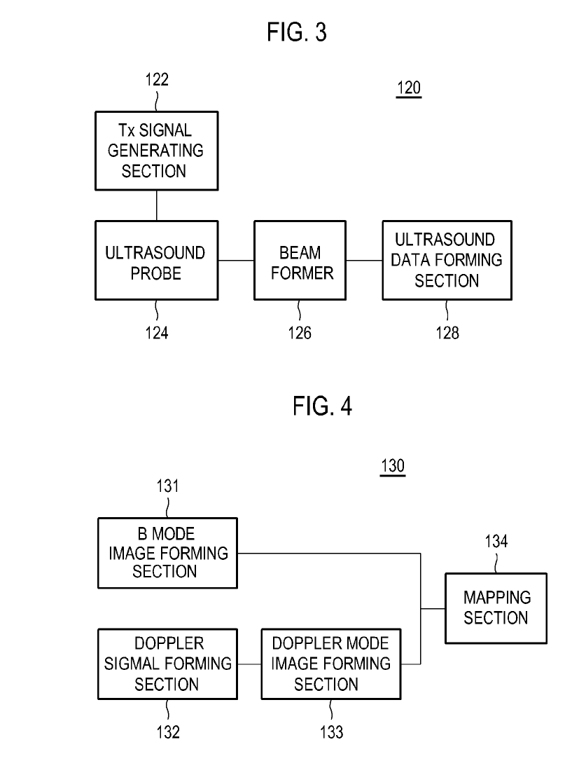

FIG. 3 is a block diagram showing an illustrative embodiment of an ultrasound data acquisition unit 120. The ultrasound data acquisition unit 120 may include a transmit (Tx) signal generating section 122, an ultrasound probe 124 having a plurality of transducer elements (not shown), a beam former 126 and a ultrasound data forming section 128.

The Tx signal generating section 122 may be configured to generate Tx signals. The Tx signal generating section 122 may generate the Tx signals and apply delays to the Tx signals in consideration of distances between the respective transducer elements and focal points. In one embodiment, the ultrasound data acquisition unit 120 may generate a first Tx signal for acquiring the B mode image 210 and a second Tx signal for acquiring a three dimensional C mode image of the ROI 220. A process of forming the three dimensional C mode image will be described in detail later.

The ultrasound probe 124 may include the plurality of transducer elements for reciprocally converting between electrical signals and ultrasound signals. The ultrasound probe 124 may transmit ultrasound signals to the target object in response to the Tx signals provided from the Tx signal generating section 122. The ultrasound probe 124 may receive ultrasound echo signals reflected from the target object to thereby output the received signals. The received signals may be analog signals. In one embodiment, the ultrasound probe 124 may transmit ultrasound signals to the target object in response to the first Tx signals provided from the Tx signal generating section 122 and receive ultrasound echo signals reflected from the target object to thereby output a first received signals. Furthermore, the ultrasound probe 124 may transmit ultrasound signals to the target object in response to the second Tx signals provided from the Tx signal generating section 122 and receive ultrasound echo signals reflected from the target object to thereby output a second received signals. The ultrasound probe 124 may include a three dimensional mechanical probe, a 2D array probe and the like. However, it should be noted herein that the ultrasound probe 124 may not be limited thereto.

The beam former 126 may convert the received signals provided from the ultrasound probe 124 into digital signals. The beam former 126 may apply delays to the digital signals in consideration of distances between the transducer elements and focal points to thereby output digital receive-focused signals. In one embodiment, the beam former 126 may form first digital receive-focused signals in response to the first received signals provided from the ultrasound probe 124 and second digital receive-focused signals in response to the second received signals provided form the ultrasound probe 124.

The ultrasound data forming section 128 may form the ultrasound data by using the digital receive-focused signals. In one embodiment, the ultrasound data forming section 128 may form a first ultrasound data (the B mode ultrasound data) in response to the first digital receive-focused signals provided from the beam former 126. The first ultrasound data may include radio frequency (RF) data. However, it should be noted herein that the first ultrasound data may not be limited thereto. Furthermore, the ultrasound data forming section 128 may form a second ultrasound data (the C mode ultrasound data) in response to the second digital receive-focused signals provided from the beam former 126. The second ultrasound data may include in-phase/quadrature (IQ) data. However, it should be noted herein that the second ultrasound data may not be limited thereto.

Referring back to FIG. 1, the processor 130 may be coupled to the user interface 110 and the ultrasound data acquisition unit 120. FIG. 4 is a block diagram showing an illustrative embodiment of the processor 130. Referring to FIG. 4, the processor 130 may include a B mode image forming section 131, a Doppler signal forming section 132, a Doppler mode image forming section 133 and a mapping section 134.

The B mode image forming section 131 may perform signal processing on the first ultrasound data to thereby form the B mode image. The signal processing may include envelope detection, log compression and the like.

The Doppler signal forming section 132 may form Doppler signals based on the second ultrasound data provided from the ultrasound data acquisition unit 120. In one embodiment, the Doppler signal forming section 122 may perform autocorrelation on the second ultrasound data to thereby form the Doppler signals including direction information on a moving direction of the target object, velocity information on a moving velocity of the target object and power information on a power intensity of the moving target object. However, it should be noted herein that the method of forming the Doppler signals in the Doppler signal forming section 132 may not be limited thereto.

The Doppler mode image forming section 133 may form a three dimensional Doppler mode image based on the Doppler signals provided from the Doppler signal forming section 132. In one embodiment, the Doppler mode image forming section 133 may form the three dimensional Doppler mode image in which the moving direction of the target object is represented by color tones based on the direction information, the velocity of the target object is represented by brightness or chroma of colors based on the velocity information and the power intensity of the target object is represented by a power profile based on the power information.

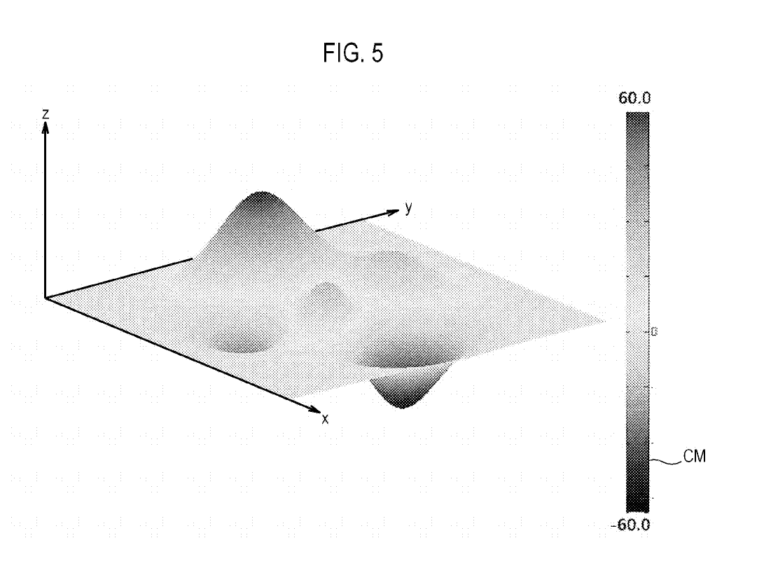

FIG. 5 is a schematic diagram showing an example of the three dimensional Doppler mode image. Referring to FIG. 5, a reference symbol CM represents a color map. The x axis represents a lateral direction, the y axis represents an elevational direction and the z axis represents the power intensity, moving direction or moving velocity of the target object. The Doppler mode image forming section 133 may form the three dimensional Doppler mode image in which the moving direction of the target object toward the ultrasound probe 124 is represented by red color tones, the moving direction of the target object away from the ultrasound probe 124 is represented by blue color tones, faster velocity of the target object than a reference velocity is represented by dark colors and slower velocity of the target object than the reference velocity is represented by bright colors. Furthermore, the Doppler mode image forming section 133 may form the three dimensional Doppler mode image in which the power intensity of the target object toward the ultrasound probe 124 (+ direction of the power intensity) is represented by protruded degree and the power intensity of the target object away from the ultrasound probe 124 (− direction of the power intensity) is represented by dented degree. The power profile represents the protruded or dented degree according to the power intensity of the target object.

The mapping section 134 may map the three dimensional Doppler mode image provided from the Doppler mode image forming section 133 onto the B mode image provided from the B mode image forming section 131 to thereby form a mapping image. That is, the mapping section 134 may map the three dimensional Doppler mode image onto the ROI 220 of the B mode image 210 to thereby form the mapping image. The methods of mapping the images are well known in the art. Thus, they have not been described in detail so as not to unnecessarily obscure the present disclosure.

Referring back to FIG. 1, the display unit 140 may display the mapping image formed by the processor 130. Furthermore, the display unit 140 may display the B mode image and the three dimensional Doppler mode image formed by the processor 130. The display unit 140 may include a cathode ray tube (CRT) display, a liquid crystal display (LCD), an organic light emitting diodes (OLED) display and the like.

In one embodiment, instructions for performing the above method of providing the three dimensional color Doppler image may be recorded in a computer readable medium using computer-readable instructions. The computer readable medium may include any type of record media, which can be read by a computer system. The computer readable medium may include read only memory (ROM), random access memory (RAM), CD-ROM, magnetic tape, floppy disk, optical-data recording apparatus and the like. The computer readable medium comprises instructions that, when executed by a processor performs the three dimensional color Doppler image providing method of an ultrasound system, cause the processor to perform the following steps: transmitting and receiving ultrasound signals to and from a target object to output ultrasound data; forming Doppler signals including power information of the target object and at least one of direction information and velocity information of the target object by using the ultrasound data; forming a three dimensional Doppler mode image representing the power information and at least one of the direction information and the velocity information by using the Doppler signals; performing a signal processing on the ultrasound data to thereby form a B mode image; and mapping the three dimensional Doppler mode image onto the B mode image to thereby form a mapping image.

Any reference in this specification to “one embodiment,” “an embodiment,” “example embodiment,” “illustrative embodiment,” etc. means that a particular feature, structure or characteristic described in connection with the embodiment is included in at least one embodiment of the present invention. The appearances of such phrases in various places in the specification are not necessarily all referring to the same embodiment. Further, when a particular feature, structure or characteristic is described in connection with any embodiment, it is submitted that it is within the purview of one skilled in the art to affect such feature, structure or characteristic in connection with other embodiments.

Although embodiments have been described with reference to a number of illustrative embodiments thereof, it should be understood that numerous other modifications and embodiments can be devised by those skilled in the art that will fall within the spirit and scope of the principles of this disclosure. More particularly, numerous variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. An ultrasound system, comprising:
an ultrasound data acquisition unit configured to transmit and receive ultrasound signals to and from a target object to output ultrasound data; and
a processor coupled to the ultrasound data acquisition unit and being configured to form Doppler signals including power information of the target object and at least one of direction information and velocity information of the target object by using the ultrasound data, the processor being further configured to form a three dimensional Doppler mode image representing the power information and at least one of the direction information and the velocity information by using the Doppler signals.

2. The ultrasound system of claim 1, wherein the processor being configured to:
represent a moving direction of the target object based on the direction information;
represent a moving velocity of the target object based on the velocity information; and
represent a power intensity of the target object based on the power information to form the three dimensional Doppler mode image.

3. The ultrasound system of claim 2, wherein the processor being further configured to:
represent the moving direction of the target object as color tones;
represent the moving velocity of the target object as a brightness or chroma of color; and
represent the power intensity of the target object as a power profile.

4. The ultrasound system of claim 3, wherein the power profile represents the power intensity of the target object as protruded or dented degree according to the power intensity of the target object.

5. The ultrasound system of claim 1, wherein the processor comprises:
a B mode image forming section configured to perform a signal processing on the ultrasound data to thereby form a B mode image;
a Doppler signal forming section configured to form Doppler signals in response to the ultrasound data provided from the ultrasound data acquisition unit;
a Doppler mode image forming section configured to form the three dimensional Doppler mode image based on the Doppler signals provided from the Doppler signal forming section; and
a mapping section configured to map the three dimensional Doppler mode image provided from the Doppler mode image forming section onto the B mode image provided from the B mode image forming section to thereby form a mapping image.

6. A method of providing a three dimensional color Doppler image, comprising:
a) transmitting and receiving ultrasound signals to and from a target object to output ultrasound data;
b) forming Doppler signals including power information of the target object and at least one of direction information and velocity information of the target object by using the ultrasound data; and
c) forming a three dimensional Doppler mode image representing the power information and at least one of the direction information and the velocity information by using the Doppler signals.

7. The method of claim 6, wherein the step c) comprises:
representing a moving direction of the target object based on the direction information;
representing a moving velocity of the target object based on the velocity information; and
representing a power intensity of the target object based on the power information to form the three dimensional Doppler mode image.

8. The method of claim 7, wherein the step c) comprises:
representing the moving direction of the target object as color tones;
representing the moving velocity of the target object as a brightness or chroma of color; and
representing the power intensity of the target object as a power profile.

9. The method of claim 8, wherein the power profile represents the power intensity of the target object as protruded or dented degree according to the power intensity of the target object.

10. The method of claim 6, further comprising:
d) performing a signal processing on the ultrasound data to thereby form a B mode image; and
e) mapping the three dimensional Doppler mode image onto the B mode image to thereby form a mapping image.

11. A computer readable medium comprising computer executable instructions configured to perform following acts:
a) transmitting and receiving ultrasound signals to and from a target object to output ultrasound data;
b) forming Doppler signals including power information of the target object and at least one of direction information and velocity information of the target object by using the ultrasound data; and
c) forming a three dimensional Doppler mode image representing the power information and at least one of the direction information and the velocity information by using the Doppler signals.

12. The computer readable medium of claim 11, wherein the computer executable instructions being further configured to perform following acts:
d) performing a signal processing on the ultrasound data to thereby form a B mode image; and
e) mapping the three dimensional Doppler mode image onto the B mode image to thereby form a mapping image.
